(19) United States
(12) Patent Application Publication
(10) Pub. No.: US 2007/0078422 A1
(43) Pub. Date: Apr. 5, 2007

(54) Absorbent article configured for controlled deformation and method of making the same

(75) Inventors: Frank S. Glaug, Chester Springs, PA (US); Jocelyn P. Rice, Philadelphia, PA (US)

Correspondence Address: RATNERPRESTIA, P O BOX 980, VALLEY FORGE, PA, 19482-0980, US

(73) Assignee: Tyco Healthcare Retail Services AG, Schaffhausen (CH)

(21) Appl. No.: 11/240,762

(22) Filed: Sep. 30, 2005

(51) Int. Cl. A61F 13/15

(52) U.S. Cl. 604/380; 604/385.01

(57) ABSTRACT

This invention provides an absorbent article configured for controlled deformation when worn by a user. The absorbent article includes a cover positionable adjacent the user, a barrier layer coupled to the cover and an absorbent layer interposed between the cover and the barrier layer. The absorbent layer of the present invention further defines at least two elongated gaps extending in a longitudinal direction, the gaps being laterally spaced on opposed sides of a centerline of the absorbent article, where each of the gaps defines a deformation zone to facilitate controlled deformation.

### FIELD OF THE INVENTION

This invention relates to absorbent articles. More particularly, it relates to absorbent articles having an absorbent article configured for controlled deformation.

### BACKGROUND OF THE INVENTION

Absorbent articles such as disposable diapers, training pants, adult incontinence garments and the like are known, their major function being to absorb and contain body exudates. Such articles are thus intended to prevent the soiling, wetting, or other contamination of clothing or other articles, such as bedding, that come into contact with the wearer. In the case of disposable diapers, for example, they are optionally provided with a basic structure that includes a liquid permeable cover, a liquid impermeable barrier layer, an absorbent layer positioned between the cover and the barrier layer, and a fluid distribution layer for distributing the liquid more uniformly over the absorbent layer, positioned between the cover and the absorbent layer.

While many developments have been made in the art of absorbent articles to improve performance and fit, there remains a need for further improvements in such absorbent articles.

### SUMMARY OF THE INVENTION

In one aspect, the invention provides an absorbent article configured for controlled deformation when worn by a user, including a cover positionable adjacent the user, a barrier layer coupled to said cover and an absorbent layer interposed between the barrier layer and the cover. The absorbent layer has at least two elongated gaps extending in a longitudinal direction. The elongated gaps are laterally spaced and positioned on opposed sides of a centerline of the absorbent layer, whereby each of the gaps defines a deformation zone to facilitate controlled deformation of the absorbent article.

In another aspect, the invention provides a method of donning an absorbent article on a user. The method includes the step of deforming the absorbent article along at least two elongated gaps extending in a longitudinal direction of an absorbent layer, thereby producing a body-conforming configuration. Additionally, the method includes the step of fastening front and back portions of the absorbent article about a waist of the user.

In a further aspect, the invention provides a method of making an absorbent article configured for controlled deformation when worn by a user. The method includes the step of defining at least two elongated gaps extending in a longitudinal direction of an absorbent layer. The gaps are laterally spaced on opposed sides of a centerline of the absorbent layer and thereby define deformation zones in the absorbent article to facilitate controlled deformation. The method also includes the step of interposing the absorbent layer between a cover and a barrier layer.

### BRIEF DESCRIPTION OF THE DRAWINGS

The invention is best understood from the following detailed description when read in connection with the accompanying drawing. It is emphasized that, according to common practice, the various features of the drawing are not to scale. On the contrary, the dimensions of the various features are arbitrarily expanded or reduced for clarity. Included in the drawing are the following figures:

FIG. 1A is a top view illustration of an exemplary embodiment of an absorbent article according to one aspect of this invention.

FIG. 1B is a cross-sectional view of the absorbent article of FIG. 1A along lines 1B-1B.

FIG. 1C is a cross-sectional view of the absorbent article of FIG. 1A along lines 1C-1C.

FIG. 2 is a top view illustration of another exemplary embodiment of an absorbent article according to an aspect of this invention.

FIG. 3 is a top view illustration of still another exemplary embodiment of an absorbent article according to an aspect of this invention.

FIG. 4 is a top view illustration of still yet another embodiment of an absorbent article according to an aspect of this invention.

### DETAILED DESCRIPTION OF THE INVENTION

The invention is best understood from the following detailed description when read in connection with the accompanying drawing, which shows exemplary embodiments of the invention selected for illustrative purposes. The invention will be illustrated with reference to the Figures. Such Figures are intended to be illustrative rather than limiting and are included herewith to facilitate the explanation of the present invention.

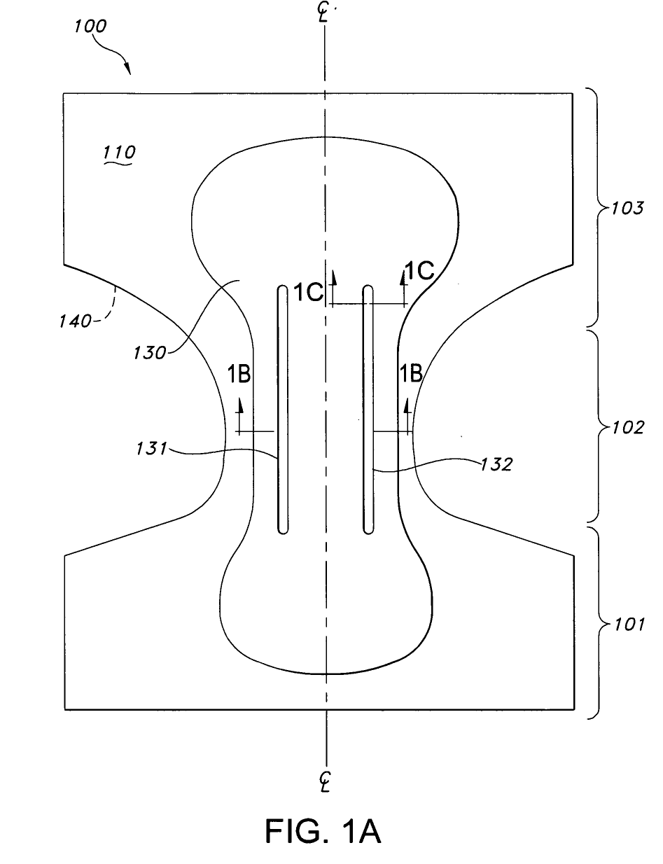

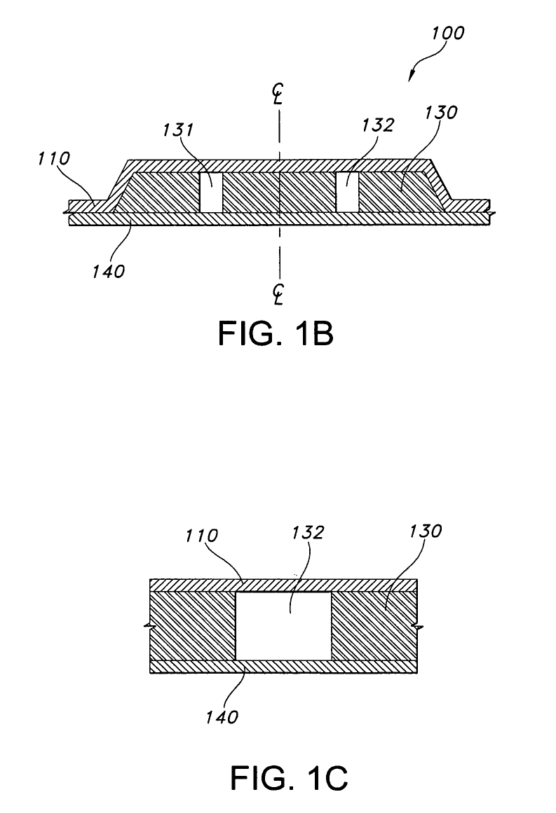

In the exemplary embodiments of the invention selected for illustration in FIGS. 1A-4, the absorbent articles 100, 200, 300 and 400 are in the form of a diaper. While the following description focuses on diapers with reference to the illustrated embodiments, it should be clear that the subject invention can be used for any type of absorbent article or garment to be worn by a person for trapping urine or menses, for example.

Certain non-limiting aspects of the invention will now be described, with general reference to the Figures.

Referring generally to the drawing (specifically, FIGS. 1A-4), illustrated embodiments of the present invention provide an absorbent article with an improved conforming-to-the-body anatomical configuration allowing controlled deformation of the absorbent core. With reference to FIG. 1A, an absorbent article 100, shown as an unfolded diaper or adult brief, includes a chassis having a front portion 101, a rear portion 103, and a crotch portion 102 positioned between the front and rear portions. The absorbent article 100 is further provided with a cover 110 positionable adjacent the user, a barrier layer 140 coupled to the cover 110, and an absorbent layer 130 interposed therebetween. The absorbent article 100 further includes at least two elongated and longitudinally positioned gaps 131 and 132 disposed in absorbent layer 130, whereby gaps 131 and 132 extend toward or into the front portion 101, through the crotch portion 102, and toward or into the rear portion 103 (but not necessarily extending the entire longitudinal length of the absorbent article 100 or the absorbent layer 130 of the absorbent article 100).

Optionally, a fluid distribution layer (not shown) may also be interposed between cover 110 and absorbent layer 130. Generally, a fluid distribution layer (not shown) serves to manage, transport, accommodate and/or direct high volumes and flow rates of fluid into the core. The fluid distribution layer can be of any type construction, e.g., a thru-air bonded/carded web, a spunbond bicomponent non-woven web, a web of crosslink cellulosic fibers, apertured 3D (three dimensional) film, adhesive bonded fibers, or the like. The fluid distribution layer (not shown) may be a single layer or may comprise multiple components functioning as an acquisition system.

One particular suitable material that can be used for the fluid distribution layer is available from PGI Nonwovens, Landisville, N.J., and has an overall basis weight of about 40 gsm, with high denier (about 10 denier) bi-component fibers situated on the top and low denier (about 6 denier) bi-component fibers situated on the bottom. The bi-component fibers are optionally made of a polypropylene inner core and polyethylene outer sheath. Preferably, the material used should be nonabsorbent and should permit the passage of liquid, but it may include hydrophilic fibers such as pulp within the interstices of the material.

Another exemplary fluid distribution layer (not shown) may be formed from a liquid permeable film such as a 3-D apertured poly sheet comprising conical holes, available from Tredegar, located in Richmond, Va. Other substrate materials are contemplated as well.

Cover 110 of absorbent article 100 is intended to be positioned proximal to the user's skin. Cover 110 is liquid permeable, allowing liquid to pass through to the rest of absorbent article 100. Cover 110 is preferably compliant, soft feeling and non-irritating to the user's skin. Cover 110 can be made from any of a number of materials known in the art, including for example, fibrous materials. The cover 110 may be made from a nonwoven material, which may be thermoplastic fibers or filaments, for example. Shape-retaining nonwoven fabrics are well known and are made by a variety of processes from fibers of polyolefins and polyesters. Where the fibers used are incapable of absorbing liquids, they may be treated with a surfactant for improved wettability. The material selected for the cover 110 may be porous to allow rapid passage of liquid. An example of one suitable material is heat bonded or point bonded nonwoven material comprising polypropylene fibers.

Cover 110 can be made from any of the materials conventional for this type of use, for example spunbonded polypropylene or polyethylene, polyester, RAYON, Hydrofil® nylon fiber available from Allied Fibers, or the like. One suitable material is a hydrophilic 15 gsm spunbond polypropylene nonwoven from Avgol Nonwoven Industries, located in Holon, Israel. Another is a 17 gsm wettable nonwoven coverstock, made of thermal bond polypropylene, available from PGI Nonwovens, Landisville, N.J.

Other non-limiting examples of suitable materials that can be used as cover 110 are woven and nonwoven polyester, polypropylene, polyethylene, NYLON, and RAYON and formed thermoplastic films, as described, for example, in U.S. Pat. No. 4,324,246 to Mullane and Smith and U.S. Pat. No. 4,342,314 to Radel and Thompson, both of which patents are incorporated herein by reference. Formed films may be selected for cover 110 because they are permeable to liquids and yet non-absorbent. Thus, the surface of the formed film, which is in contact with the body, remains substantially dry and is more comfortable to the wearer.

Cover 110 may be adhesively secured in place by any suitable construction adhesive or hydrophilic adhesive, such as cycloflex adhesive available from National Starch and Chemical, Bridgewater, N.J.

Barrier layer 140 is positioned on the opposite side of absorbent layer 130 from the cover 110. Barrier layer 140 is the portion of the absorbent article 100 that is distal or opposite from the user's skin. Barrier layer 140 is preferably a liquid impermeable material such as a poly blend. Barrier layer 140 is proximal to, or in some embodiments attached to, clothing such as an undergarment in use. Barrier 140 blocks the passage of any unabsorbed liquid from absorbent article 100 and provides support for the absorbent layer. Exemplary features of the absorbent layer 130, which includes at least two elongated gaps 131 and 132, will be described hereinafter in greater detail.

Materials suitable for use in forming barrier 140, which is configured to prevent the passage of liquid, are well known in the industry. Such materials include, for example, films such as polyethylene, polypropylene, and copolymers, as are known in the absorbent article art. Suitable materials may include for example a liquid-impermeable laminate comprising a soft nonwoven (cloth-like/hydrophobic) on the outside and fluid-impermeable film (low gauge poly) on the inside. An example of this is a poly laminate available from Clopay Plastic Products Company, Cincinnati, Ohio, which consists of 0.6 mil polyethylene film and 17 gsm (gram per square meter) SMS (spunbond/meltblown/spunbond) nonwoven. Another version is a poly laminate 9B-396 available from Pliant Corporation of Newport News, VA, which consists of 0.3 mil copolymer film and 14 gsm SBPP (spunbond polypropylene) nonwoven. However, other laminate variations may be used in various gauges and basis weights. For instance, other polymers (polypropylene, olefins, polyester, co-extruded polymers, etc.) or coatings (adhesive, synthetic rubber, latex, polyurethane, etc.) can be used in place of the polyethylene film. Other material components (polypropylene, polyethylene, bi-component fibers, polyester, cotton, RAYON, NYLON, olefins, etc.) can be used in either woven or nonwoven (spunbond, thermal bond, through-air bond, etc.) construction in place of the SMS outer cover. The preferred fluid-impermeable film for the liquid-impermeable laminate is a breathable 0.8 mil polyethylene version, which contains calcium carbonate, available from Tredegar Film Products, Richmond, Va. This material allows water vapor to pass through it, but does not permit the liquid itself to pass through it.

Absorbent article 100 further comprises absorbent layer 130 which may be formed from an absorbent material such as an airlaid. Further, the absorbent material may include Super Absorbent Polymer (SAP), in which the SAP optionally has a basis weight in the range of about 300 to about 500 gsm. Still further, the airlaid may have a SAP concentration of about 50% by weight.

The term SAP as used herein encompasses a hydrocolloid material, which is capable of absorbing many times its own weight of aqueous liquid. These materials are generally prepared by polymerizing one or more monomers, which if homopolymerized by conventional methods, would form water-soluble polymers. To render them water insoluble, these polymers or mixtures of them are typically crosslinked. Known polymers of this type are based on cross-linked salts of polyacrylic acid or polymethacrylic acid. Exemplary superabsorbent materials suitable for use include polyacrylamides, polyvinyl alcohol, ethylene maleic anhydride, and the like. Preferred are SAP's comprising crosslinked salts of polyacrylic acid.

The SAP may have a relatively uniform particle size, or may have a distribution of particle sizes. An exemplary form of SAP is a granular or powdered material having a distribution of particle sizes ranging from about 45 μm to about 850 μm, preferably between about 106 μm and about 850 μm. The presence of some proportion of particles of small size may encourage effective penetration of such particles into the absorbent layer 130, and may also increase the rate of liquid uptake when the absorbent article receives a liquid insult, due to the high surface area per unit weight of small particles.

The absorbent layer 130 is interposed between the cover 110 and the barrier layer 140. The barrier layer 140 can be maintained in contact with the absorbent layer 130 by applying adhesive, optionally in spaced, limited areas, to an inner surface of the barrier layer 140. Additionally, cover 110 may be adhered to barrier layer 140 by the application of an adhesive material at a location corresponding to elongated gaps 131 and 132.

Examples of suitable adhesives used for this purpose include the acrylic emulsion E-1833BT manufactured by Rohm and Haas Company of Philadelphia, Pa. and acrylic emulsions manufactured by H. B. Fuller Company of St. Paul, Minn. Additionally, water-absorbing adhesives may be used, such as are known in the art. Also contemplated are thermoplastic hot melt adhesives such as 34-563A, available from National Starch, Inc.

Although the absorbent article embodiment illustrated in FIG. 1A includes an absorbent layer, a cover, and a barrier layer, an absorbent article according to this invention may include fewer or more components. For example, an absorbent article is optionally provided with a fluid distribution layer, fastening mechanisms, elastic components, gathers, tabs, and other components depending on the intended use of the article.

Referring now specifically to FIG. 1A, that figure illustrates one embodiment, including cover 110, barrier layer 140, and absorbent layer 130. As shown, the absorbent article has a front portion 101, a back portion 103 and a crotch portion 102 positioned therebetween. The absorbent layer 130 has a centerline CL running longitudinally along the length of the absorbent article 100. Elongated gaps 131 and 132 extend in the longitudinal direction parallel to the centerline from the front portion 101 to the rear portion 103 of the absorbent article 100. The elongated gaps 131 and 132 are laterally spaced (and optionally equidistantly spaced) on opposed sides of the centerline.

Each of gaps 131 and 132 define a deformation zone to facilitate controlled deformation of the absorbent article when worn by the user. In other words, when donned by a user, the absorbent article will tend to deform in a controlled manner generally along the gaps 131 and 132. Such deformation is likely to take the form of a bend or partial fold, with a crease or curvature generally extending along the gaps 131, 132. It has been discovered that the controlled or directed deformation of the absorbent article along regions such as gaps 131, 132 helps to promote a body conforming fit of the absorbent article when donned. Such fit contributes to improved performance (e.g., leak resistance), improved comfort, and improved conformance to clothing.

FIG. 1B is a cross-sectional view of the absorbent article of FIG. 1A along line 1B-1B, illustrating topsheet 110, barrier layer 140, and absorbent layer 130 interposed therebetween and having elongated gaps 131 and 132 extending therethrough. FIG. 1C is a cross-sectional view of FIG. 1A along line 1C-1C. FIG. 1C illustrates topsheet 110 and barrier layer 140, with absorbent layer 130 interposed therebetween. As shown, elongated gap 132 extends from topsheet 110 completely through absorbent layer 130 to barrier layer 140. It is contemplated that elongated gaps 131 (not shown in FIG. 1C) and 132 need not extend the entire thickness of absorbent layer 130, but need only be of sufficient depth to allow the absorbent article to be configured to be bent or folded or otherwise deformed to allow controlled deformation of the absorbent article.

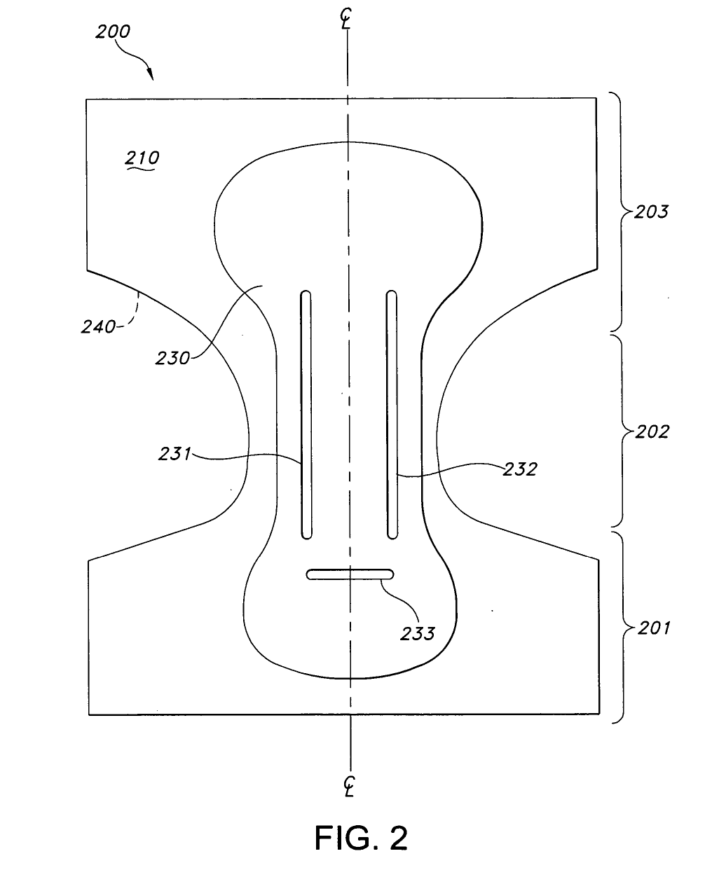

In another embodiment of the present invention, as exemplified in FIG. 2, the absorbent article may also be provided with an elongated gap located in a transverse direction of the absorbent article. In this embodiment, absorbent article 200 has a front portion 201, a back portion 203, and a crotch portion 202, with absorbent layer 230 interposed between cover 210 and barrier layer 240. As in the embodiment shown in FIG. 1A, the absorbent layer has a centerline CL running longitudinally along the length of the absorbent article. Elongated gaps 231 and 232 extend in the longitudinal direction parallel to the centerline from the front portion 201 to the rear portion 203 of the absorbent article 200 with the elongated gaps 231 and 232 being laterally spaced on opposed sides of the centerline. In addition, absorbent article 200 also includes elongated gap 233 disposed transversely in front portion 201 in a region adjacent the crotch portion 202 of absorbent article 200. The location of the transverse gap shown in FIG. 2, i.e., in front portion 201 of the absorbent article 200, is optionally adjusted depending upon a variety of factors (e.g., the size of the absorbent article, the nature of the absorbent article, etc.). For example, one or more such transverse gaps can be provided in the crotch portion 202.

Each of gaps 231, 232 and 233 define a deformation zone to facilitate controlled deformation of the absorbent article when worn by the user. As discussed previously in connection with article 100, when donned by a user, the absorbent article 200 will tend to deform in a controlled manner generally along the gaps 231, 232 and 233. In this embodiment, the transverse and longitudinal gaps 231, 232 and 233 tend to cooperate with one another to improve fit, including comfort and performance.

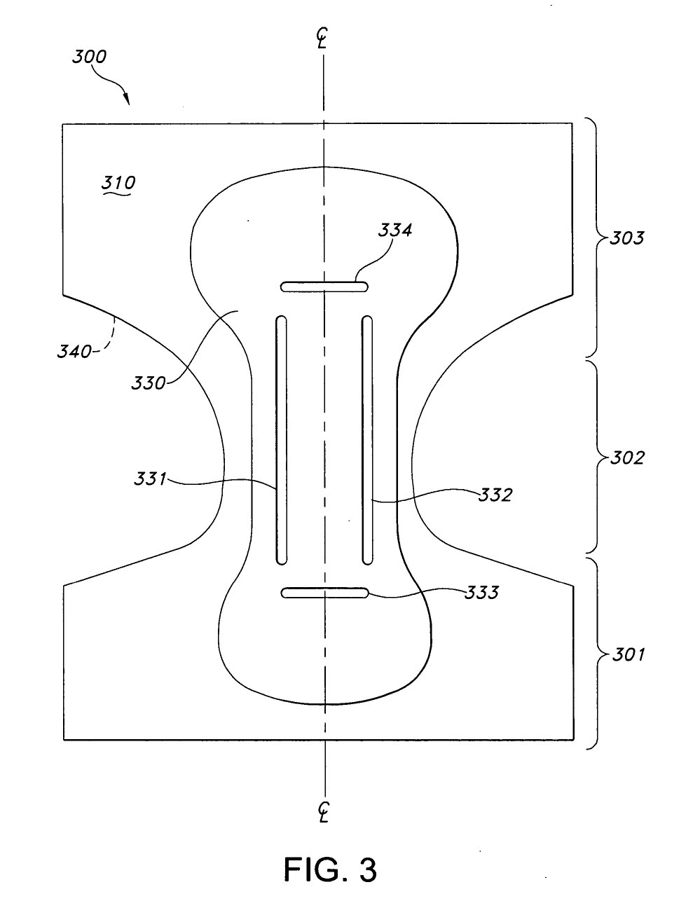

In yet another embodiment of the present invention, as exemplified in FIG. 3, the absorbent article may also be provided with two elongated gaps located in transverse directions of the absorbent article. In this embodiment, absorbent article 300 has a front portion 301, a back portion 303, and a crotch portion 302, with absorbent layer 330 interposed between cover 310 and barrier layer 340. As in the embodiment shown in FIG. 1A, the absorbent layer has a centerline running longitudinally along the length of the absorbent article. Elongated gaps 331 and 332 extend in the longitudinal direction parallel to the centerline from the front portion 301 to the rear portion 303 of the absorbent article 300, with a first elongated gap 331 and a second elongated gap 332 being laterally spaced on opposed sides of the centerline. First transverse gap 333 is located adjacent front portion 301 of absorbent article 300 (in front portion 301 as shown or in crotch portion 302) and second elongated gap 334 is disposed in the rear portion 303 of the absorbent article 300 located adjacent crotch portion 302 of absorbent article 300 (in rear portion 303 as shown or in crotch portion 302). Each of gaps 331, 332, 333 and 334 define a deformation zone to facilitate controlled deformation of the absorbent article when worn by the user. As discussed previously in connection with articles 100 and 200, when donned by a user, the absorbent article 300 will tend to deform in a controlled manner generally along the gaps 331, 332, 333 and 334. In this embodiment, the transverse and longitudinal gaps 331, 332, 333 and 334 tend to cooperate with one another to improve fit, including comfort and performance.

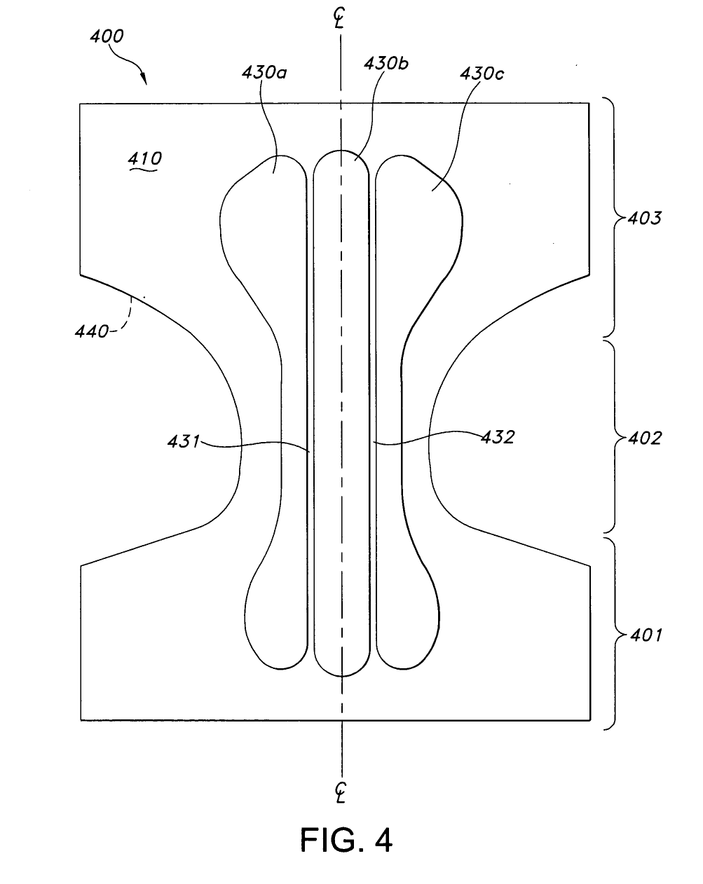

FIG. 4 is a top view illustration of another embodiment of an absorbent article according to an aspect of the invention. As exemplified in FIG. 4, absorbent article 400 of the present invention comprises a front portion 401, a rear portion 403, and a crotch portion 402. In addition to cover 410, which is positionable adjacent the user, and a barrier layer 440 coupled to cover 410, the absorbent article 400 includes an absorbent layer comprising three elongated portions 430a, 430b and 430c extending in the longitudinal direction substantially parallel to the centerline of the absorbent article. Each of the elongated portions 430a and 430c is laterally spaced from one another on opposed sides of the centerline CL, and the elongated portion 430b is substantially bisected by the centerline (e.g., the elongated portion 430b bridges the centerline and has substantially equal portions on each side of the centerline). The elongated portions 430a, 430b and 430c lie substantially parallel to one another and are not connected at the two opposing ends of each portion 430a, 430b and 430c, thereby defining channels 431 and 432 that completely separate each of the three elongated longitudinal portions 430a, 430b and 430c of the absorbent layer. As discussed previously in connection with articles 100, 200, and 300, when donned by a user, the absorbent article 400 will tend to deform in a controlled manner generally along the gaps 431 and 432. In this embodiment, the longitudinally extending gaps 431 and 432 tend to cooperate with one another to improve comfort and performance.

Though the configurations illustrated by the Figures may optionally be selected, other shapes are contemplated as well, depending on the size of the absorbent article, the intended use for the absorbent article, and other design considerations. Also, the configurations of the respective components of the absorbent article may differ from one another. For example, although an outer perimeter of the article may be substantially hourglass shaped, the fluid distribution layer, absorbent layer, and other components may have rounded shapes or different configurations. Further, the portions of the absorbent layer that define the gaps or channels are optionally provided with the same or different shapes or sizes, depending on specific design criteria.

The present invention also provides a method of putting on, or donning, the absorbent article. When donning the absorbent article, the user deforms the absorbent article along the elongated gaps, which extend in a longitudinal direction of an absorbent layer. Through this action, the user achieves controlled deformation of the absorbent article to produce an improved body-conforming configuration that in turn provides improved absorbency performance and fit. In another embodiment, along with deforming the article along the longitudinal direction, the user may also deform the absorbent article transversely along a gap located in the front portion of the absorbent article, along a transverse gap located in the rear portion of the absorbent article, or along transverse gaps located in each of the front and rear portions of the absorbent article. When donning the absorbent article, the cover is positioned adjacent the user, while the barrier layer is thus oriented away from or distal the user. In each of these embodiments, if fastening mechanisms are provided, the user then fastens the front and back portions of the absorbent article about a waist of the user, using the fastening mechanisms provided with the absorbent article.

The fastening mechanisms can be any fastening mechanisms known in the art. These may include well-known pin fasteners, or more recent innovations including fasteners made of filamentary material, such as hook and loop filamentary materials manufactured by Velcro Corporation and Aplix Corporation. In this regard, reference is made to U.S. Pat. No. 4,537,591 to Coates, incorporated herein by reference, which discloses a cloth diaper having filamentary fasteners together with a self-closing tab cover that protects the fasteners from buildup of lint during washing.

Whether for reusable or disposable products, various fastening systems have been employed for fastening the absorbent products to the wearer or to the clothing of the wearer. For example, the waistband of a diaper is preferably fastened around the waist of the wearer, and the fastening system is generally intended to hold the diaper in snug encircling fashion on the wearer's torso. After the diaper is soiled, it is removed by unfastening the tabs, thereby opening the waist.

The present invention also provides a method of making an absorbent article configured for controlled deformation when worn by a user. One embodiment of such a method comprises interposing an absorbent layer between a cover and a barrier layer, defining at least two elongated gaps extending in a longitudinal direction of the absorbent layer, the gaps being laterally spaced on opposed sides of a centerline of the absorbent layer, thereby defining deformation zones to facilitate the controlled deformation. In another embodiment of the present invention, in addition to the aforementioned steps, the method may further include interposing a fluid distribution layer between the cover and the absorbent layer. In yet another embodiment, the method may also comprise defining at least one gap transverse to the longitudinal direction and located in the front portion or, alternatively, in the rear portion of the absorbent article. In still yet another embodiment the method further includes defining at least one gap transverse to the longitudinal direction in each of the front and rear portions of the absorbent articles.

While preferred embodiments of the invention have been shown and described herein, it will be understood that such embodiments are provided by way of example only. For example, absorbent articles according to the invention may be used in a variety of absorbent articles, including for example diapers, adult incontinence pads, and feminine hygiene products. Numerous variations, changes and substitutions will occur to those skilled in the art without departing from the spirit of the invention. Accordingly, it is intended that the appended claims cover all such variations as fall within the spirit and scope of the invention.

What is claimed:

1. An absorbent article configured for controlled deformation when worn by a user, said absorbent article comprising:
a cover positionable adjacent the user;
a barrier layer coupled to said cover; and
an absorbent layer interposed between said cover and said barrier layer, said absorbent layer having at least two elongated gaps extending in a longitudinal direction and laterally spaced on opposed sides of a centerline of said absorbent layer, each of said gaps defining a deformation zone to facilitate said controlled deformation.

2. The absorbent article of claim 1, further comprising a fluid distribution layer interposed between said cover and said barrier layer.

3. The absorbent article of claim 1, said absorbent layer defining at least a first gap transverse to said longitudinal direction.

4. The absorbent article of claim 3, wherein said first gap is disposed in a front or a rear portion of said absorbent article and adjacent a crotch portion of said absorbent article.

5. The absorbent article of claim 4, further comprising a second gap disposed transverse to said longitudinal direction, wherein said second gap is disposed in a front or a rear portion opposite said first gap and adjacent said crotch portion of the absorbent article.

6. The absorbent article of claim 1, said absorbent layer comprising at least three elongated portions together defining said elongated gaps.

7. The absorbent article of claim 6, wherein said elongated gaps are channels.

8. The absorbent article of claim 1, said absorbent layer having only two elongated gaps extending in said longitudinal direction, each of said gaps defining a deformation zone to facilitate said controlled deformation.

9. The absorbent article of claim 8, said elongated gaps being spaced the same distance from said centerline of said absorbent layer.

10. The absorbent article of claim 1, at least one of said elongated gaps extending along a full length of said absorbent layer.

11. The absorbent article of claim 10, each of said elongated gaps extending along the full length of said absorbent layer.

12. The absorbent article of claim 10, said at least one elongated gap having open ends.

13. The absorbent article of claim 1, at least one of said elongated gaps extending less than a full length of said absorbent layer.

14. The absorbent article of claim 13, each of said elongated gaps extending less than the full length of said absorbent layer.

15. The absorbent article of claim 13, said at least one elongated gap having closed ends.

16. A method of donning an absorbent article on a user comprising the steps of:
deforming the absorbent article along at least two elongated gaps extending in a longitudinal direction of an absorbent layer, thereby producing a body-conforming configuration; and
fastening front and back portions of the absorbent article about a waist of the user.

17. The method of claim 16, said deforming step further comprising deforming the absorbent article along at least one gap in the absorbent layer transverse to the longitudinal direction of the absorbent layer.

18. The method of claim 17, said deforming step further comprising deforming the absorbent article along at least two gaps in the absorbent layer transverse to the longitudinal direction of the absorbent layer.

19. A method of making an absorbent article configured for controlled deformation when worn by a user, said method comprising the steps of:
defining at least two elongated gaps extending in a longitudinal direction of an absorbent layer and laterally spaced on opposed sides of a centerline of the absorbent layer, thereby forming deformation zones to facilitate the controlled deformation; and
interposing the absorbent layer between a cover and a barrier layer.

20. The method of claim 19, further comprising the step of interposing a fluid distribution layer between the cover and the barrier layer.

21. The method of claim 19, said defining step further comprising defining at least a first gap transverse to the longitudinal direction of the absorbent layer.

22. The method of claim 21, said defining step further comprising disposing the first gap in a front or a rear portion of the absorbent article and adjacent a crotch portion of the absorbent article.

23. The method of claim 22, said defining step further comprising disposing a second gap transverse to the longitudinal direction in a front or a rear portion opposite the first gap and adjacent the crotch portion of the absorbent article.

24. The method of claim 19, said defining step comprising defining only two elongated gaps extending in the longitudinal direction, each of the gaps forming a deformation zone to facilitate the controlled deformation.

25. The method of claim 24, said defining step comprising spacing the elongated gaps the same distance from the centerline of the absorbent layer.

26. The method of claim 19, said defining step comprising extending at least one of the elongated gaps along a full length of the absorbent layer.

27. The method of claim 26, said defining step comprising extending each of the elongated gaps along the full length of the absorbent layer.

28. The method of claim 19, said defining step comprising extending at least one of the elongated gaps less than a full length of the absorbent layer.

29. The method of claim 28, said defining step comprising extending each of the elongated gaps less than the full length of the absorbent layer.
